(19) United States
(12) Patent Application Publication
(10) Pub. No.: US 2020/0121408 A1
(43) Pub. Date: Apr. 23, 2020

(54) Surgical Sheet

(71) Applicant: MEDENVISION bvba, Aarschot (BE)

(72) Inventors: Pieter Deboeck, Aarschot (BE); Filip Vanlimbergen, Aarschot (BE); Johan Vanhulle, Aarschot (BE); Matthew Campsteyn, Aarschot (BE); Wouter Foulon, Aarschot (BE); Kristoff Corten, Aarschot (BE); John Bohannon Mason, Charlotte, NC (US)

(21) Appl. No.: 16/655,909

(22) Filed: Oct. 17, 2019

(30) Foreign Application Priority Data
Oct. 17, 2018 (BE) 2018/5714

(51) Int. Cl. A61B 46/20

(52) U.S. Cl. CPC A61B 46/20; A61B 2046/205

(57) ABSTRACT

The current invention concerns an improved surgical drape, a method for deploying said surgical drape over a patient, a method for folding up said surgical drape for use, and a use of the surgical drape during surgery.

### TECHNICAL FIELD

The invention pertains to the technical field of surgical drapes for use in surgical procedures, as well as the use of said systems. More specifically, the present invention relates to methods and systems for shielding the surrounding area of an incision site during a surgical procedure.

### BACKGROUND

Good hygiene and a sterile environment are critical when performing surgical procedures, especially in open surgery. One of the measures taken is the covering of the patient with the aid of a surgical drape. The surgical drape ensures that parts of the body of the patient where the surgical procedure is not taking place are shielded from the incision site, and vice versa, so that the incision site does not come into contact with parts of the patient that have not been sterilized, and the risk of contamination is thus avoided or substantially reduced.

Currently, the patient is disinfected at and around the incision site to make it sterile. The area around the incision site is provided with a surgical drape. By default, a surgical drape in a standard size is used for this. The positioning of the drape and the disinfection of the patient is done by medical assistants in preparation for the surgical procedure, and often takes a long time to complete. Since this preparation needs to be done in the operating theatre, this limits the time available for surgical procedures.

The main concern the present invention addresses, is the fact that for a number of operating tables, on one or both of the lateral sides thereof, a movable ‘modular instrument holder’ is provided, on which instruments can be placed (in a ‘glove-hand’ connection, the instrument sliding over an elongate part of the instrument holder. This solves the pre-existing problem of keeping the instrument holder and instruments themselves separate, avoiding cross-infections. In the prior art, a part of the sheet was simply cut open or removed to allow the instrument holder and/or instrument to pass through the sheet. However, this is to be avoided for the sake of keeping both sides of the sheet separated. Another option is to simple clamp a piece of the sheet between the connection of the instrument and instrument holder. However, this can cause tearing, but mostly is not advisable as during the surgery, it is often necessary to move the instrument up and/or down, which could create tension on the sheet, which is usually affixed around the area to be operated on and/or at other points. This could potentially lead to the sheet being pulled free from the patient's body, or being moved so the incision window no longer lines up with the area to be operated on.

Lastly, it is crucial that the surgical sheets can be folded, and unfolded in practical fashions, which ensure sterility during deployment.

The above problems and objectives are addressed by the current invention.

### SUMMARY OF THE INVENTION

It is an object of embodiments of the present invention to provide good methods and products for the preparation of a patient for surgery. It should be noted that the preparatory steps required to prepare a patient are not part of the surgical method itself and consequently, the method envisaged is not a surgical method.

It is an advantage of embodiments according to the present invention that the patient can be prepared for a surgical procedure in an efficient manner. This efficiency relates not only to the fact that a very good shielding is provided, but also to the fact that the shielding can be accomplished in a quick and simple way.

It is thus an advantage of embodiments according to the present invention that time is saved when providing the necessary shielding so that generally speaking, more time becomes available in the operating theatre and, consequently, more procedures can be performed with the same infrastructure.

It is an advantage of embodiments according to the present invention that, depending on the location on the body at which the medical procedure needs to be performed, a different type of shielding can be provided.

It is an advantage of embodiments according to the present invention that the shielding can be applied in a sterile manner.

It is an advantage of some embodiments according to the present invention that fixation elements can be provided in the shielding for the attachment of surgical retractors to keep the wound open. Since the shielding is provided around the leg or the arm of the patient, in this way, the body of the patient is also used to keep the fastening elements in place during the surgical procedure, or to move them to specific positions.

It is an advantage according to embodiments of the present invention that the chance of sterilization errors is minimized, since the surgical drape as described allows installation within a few movements of the patient in a sterile way.

It is an advantage according to embodiments of this aspect of the present invention that fewer operations, less time and less manpower is needed to drape a patient.

It is an advantage according to embodiments of this aspect of the present invention that the limbs of the patient can be freely manipulated, while the risk of sterilization errors is greatly reduced.

In a first aspect, the invention provides a surgical drape consisting of a one-piece sheet having a distal surface and a proximal surface relative to a surgical patient, the proximal surface being adjacent to the patient, wherein the sheet comprises at least one transparent or open incision window at a first, internal position, which transparent or open position extends through the sheet, whereby the surgical drape is characterized in that the sheet comprises at least one through-hole, preferably circular or ellipse-shaped, through the sheet at a second, internal position, whereby said first and second position are spatially separated over a predetermined distance. Preferably, the through-hole has reinforced edges (for instance, with a thickened border, possibly reinforced with rubber and/or other elastic materials).

Preferably, the predetermined distance between the first and second position is adjusted for a specific surgery, as the location of the incision window with respect to a steady anchoring point will necessarily require different positioning depending on the type of surgery (for instance hip versus groin versus stomach, etc.). The most commonly practiced surgery for which the sheet is especially suited, is hip surgery (anterior approach more exactly), which would mean the predetermined distance is preferably at least 15 cm, preferably at least 20 cm, more preferably at least 25 cm, even more preferably at least 30 cm, and most preferably at least 35 cm or even at least 40 cm. Likewise, there are preferred maximal distances between the first and second position for this application, preferably at most 60 cm, more preferably at most 55 cm, even more preferably at most 50 cm, and most preferably at most 48 cm. As will be mentioned later, said distances are center-to-center distances.

The through-hole is hereby adapted, specifically dimensioned, to receive a first elongate element through the through-hole. Said elongate element refers to a mounting element that is commonly provided in most operating tables, allowing the operating staff to mount modular surgery instruments thereon. The through-hole therefore is dimensioned to accommodate such a mounting element, and preferably has a diameter comprised between 5 mm and 65 mm, more preferably between 15 mm and 60 mm, even more preferably between 25 mm and 55 mm, and most preferably between 35 mm and 55 mm. In some embodiments, the sheet comprises a removable cover for covering the through-hole, to be removed upon use.

In a further embodiment, the drape according to the invention comprises at least one sleevelike or pocketlike portion at said second, internal position, extending from the through-hole which defines an opening of the sleevelike or pocketlike portion, said sleevelike or pocketlike portion being extendable from said second position, whereby said first and second position are spatially separated over a predetermined distance, preferably at least 15 cm, more preferably at least 20 cm, even more preferably at least 25 cm. It is of note that the sleevelike or pocketlike portion is preferably closed at the distal end, however, embodiments wherein said sleeve- or pocketlike portion is open at said distal end are possible as well.

In a slight variation on the above further embodiment, the sleevelike or pocketlike portion is provided separately from the sheet. This would allow sterility to be maintained, by first pulling the sleeve over the mounting element of the operating table (first elongate element), after which the sheet can be positioned, whereby the through-hole is pulled over the mounting element which is already covered by the sleevelike or pocketlike portion. In an even further embodiment, the sheet and/or the sleevelike or pocketlike portions comprise fastening elements to allow said elements to be affixed to each other, further ensuring sterility.

In a further embodiment, the sheet comprises at least one anchoring zone at a third, internal position on the proximal surface of the sheet adapted for positioning, and preferably affixing, said anchoring zone on the proximal surface to a biometric anchoring point or zone on the body of the surgical patient, preferably to or around the knee or patella, whereby said third position is spatially separated from the first position over a predetermined distance, said predetermined distance between the first and third position being dependent on the biometric anchoring point or zone, and whereby said third position is spatially separated from the second position over a predetermined distance, preferably whereby the sheet at least partially transparent, translucent or open at or around the anchoring zone. The predetermined distance between the first and third position is specifically dependent on an averaged distance between the area to be operated on and the biometric anchoring point. We wish to underline that different sheets will typically be used for different types/zones of surgery (hip versus stomach versus groin, etc.), which allows the distance between the first and third point to be adapted to the specific situation at hand. Since the incision window is usually made overly large, to accommodate variations between patients, this will also allow the sheet to be used on most patients, as the dimensions of the human body do not vary too much between different patients. Therefore, if the distance between the first and third position reflects that for the ‘average’ human, the large incision window will ensure that the sheet is successfully positioned for almost any patient which does not greatly differ from the norm (for which special cases, custom-made sheets can easily be provided).

In a second aspect, the present invention provides a method for unfolding and positioning a folded surgical drape according to the invention, on a body of a surgical patient which is located on an operating table, said table comprising at least one lateral, movable, substantially vertically extending, mounting element for receiving modular surgery instruments, said method comprising the following steps:
a. positioning the incision window over the area of the body to be operated on, preferably by affixing said incision window around said area via an adhesive portion on the proximal surface of the sheet at or around the incision window;
b. placing the through-hole over the mounting element;
c. unfolding the sheet, thereby covering the body of the surgical patient;
d. optionally placing a modular surgery instrument on the mounting element;

whereby the step of positioning the incision window over the area to be operated on precedes the step of placing the through-hole over the mounting element.

Preferably, the sheet comprises at least one, preferably closed, sleevelike or pocketlike portion as discussed in this document, the method comprising a step of placing the sleevelike or pocketlike portion over the mounting element, thereby at least partially pulling said sleevelike or pocketlike portion over the length of said mounting element, whereby this step is performed during or subsequent to placing the through-hole over the mounting element.

In a third aspect, the present invention provides a method for folding up a symmetrical surgical drape as discussed further in the text, on a body of a surgical patient which is located on an operating table, said table comprising at least one lateral, movable, substantially vertically extending, mounting element for receiving modular surgery instruments, said method comprising the following steps:
a. positioning the incision window on the first lateral symmetric side of the sheet over the area of the body to be operated on, preferably by affixing said incision window around said area via an adhesive portion on the proximal surface of the sheet at or around the incision window;
b. positioning the incision window on the second lateral symmetric side of the sheet over an area on the body mirrored to the area of the body to be operated on, preferably by affixing said incision window around said mirrored area via an adhesive portion on the proximal surface of the sheet at or around the incision window;
c. placing the through-hole on the first lateral symmetric side of the sheet over the mounting element most proximal to said first lateral symmetric side of the sheet;
d. placing the through-hole on the second lateral symmetric side of the sheet over the other mounting element;
e. unfolding the sheet, thereby covering the body of the surgical patient;
f. optionally placing modular surgery instruments on one or both of the mounting elements

whereby the steps of positioning the incision window over the area to be operated on precede the steps of placing the through-hole over the mounting element.

Preferably, the sheet comprises two, preferably closed, sleevelike or pocketlike portions as discussed in this document, on each lateral symmetric side of the sheet, the method comprising a step of placing the sleevelike or pocketlike portions over the mounting element, thereby at least partially pulling said sleevelike or pocketlike portions over the length of the mounting elements, whereby these steps are performed during or subsequent to placing the through-holes over the mounting elements on the respective lateral sides.

In a preferred embodiment of the invention according to the second and third aspect, using a specific embodiment of the surgical drape comprising one or more anchoring zones, the method comprises the additional step of positioning (preferably affixing) the anchoring zone to a biometric anchoring point or zone on the body of the patient, whereby the step of positioning, preferably affixing, the anchoring zone to the biometric anchoring point or zone precedes the step of positioning the incision window over the area to be operated on.

In a preferred embodiment, the anchoring zone is a fixation zone, wherein positioning the fixation zone refers to fixating said fixation zone at the desired position on the body of the patient.

In a fourth aspect, the present invention provides a method for folding up a surgical drape according to the invention, said sheet being essentially rectangular and having a head side, a leg side, and two lateral sides, comprising the following steps:
a. folding up a section of the sheet on the lateral side of said sheet most proximal to the sleevelike or pocketlike portion, over the sheet substantially up to, but not over, the incision window, and whereby said folded section comprises said sleevelike or pocketlike portion, preferably whereby the folded section on the lateral side is accordion folded;
b. folding up a section of the sheet on the head side, preferably at least partly, more preferably at most partly, over the incision window, preferably whereby the folded section on the head side is accordion folded;
c. further folding up the sheet on the head side, whereby the further folded section comprises the entire incision window with a maximal beyond the incision window further margin of said further folded section of 20 cm;
d. folding up the section of the sheet on the leg side which is not folded over with the folded head side section or the further folded head side section, preferably whereby the folded section on the leg side is accordion folded.

It is of particular note that step c. can be left out in some embodiments, but is highly advantageous in embodiments of the sheet where the sheet comprises one or more anchoring zones.

The surgical drape according to the invention is, in a fifth aspect, particularly suitable for use in hip surgery, specifically anterior approach hip surgery.

### DESCRIPTION OF FIGURES

FIG. 1A-1N show a step-by-step method of folding an embodiment of the surgical drape of the invention shows.

FIG. 2A-2F show a number of specific folds, preferably used in the FIGS. 1A-1N.

FIG. 3A-D show a surgical drape according to an embodiment of the invention, with three detailed views of specific zones of the sheet.

FIG. 4A-4H show a surgical drape according to an embodiment of the invention, along with certain folding steps.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings: “A”, “an”, and “the” as used herein refers to both singular and plural referents unless the context clearly dictates otherwise. By way of example, “a compartment” refers to one or more than one compartment. This is particularly of note as the sheet, and associated methods are discussed as having for instance ‘a sleeve’, and ‘an incision window’, ‘an anchoring zone’, etc., although the sheet preferably would have one of each on both lateral sides of the sheet (most preferably being symmetrical). The person skilled in the art will readily recognize this from the text, and also from the figures.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

Note that when mentioning distances between certain “regions”, “portions”, “positions” or “zones” which have a certain surface area, the term “distance” refers to the distance between the approximate centers of said regions, portions, positions or zones, unless explicitly stated otherwise.

In a first aspect, the invention provides a surgical drape as described in claim 1. The advantages of said surgical drape are apparent, when taking in mind operating tables used in for instance hip surgery. These employ a mounting element upon which modular instruments can be fixed (for instance to hold the wound open, etc.) on the side of the table, for instance the Hana® table currently provided by Mizuho OSI. Specifically for the Hana® table, it should be noted that the holder is movable in height setting, thus requiring some flexibility in the surgical drape. However, existing surgical drapes are not adapted to this set-up, which leads to unsafe operating procedures, as discussed earlier. The applicant therefore improved the existing surgical drapes, which up till now where generally rectangular sheets with an incision window, and provided a pocketlike or sleevelike portion, towards a lateral side of said sheet, which was designed to fit over the elongate mounting element (instrument holder) upon which instruments can be fixed. In certain embodiments, the sleeve is not present, and only a through-hole is used, which can also be fitted over the mounting element, giving the same advantages (though the sleeve will increase sterility even further). Given the knowledge of the area to be operated on, and the type of table, the distance between the incision window and the sleevelike or pocketlike portion/through-hole can be easily estimated, which creates a reliable template for their positions on the surgical drape. The applicant for instances notes that anterior approach hip surgery on the mentioned Hana® table, which is especially suitable for said surgery, would have a distance between the sleevelike or pocketlike portion/through-hole and the incision window between 30 cm and 50 cm, preferably about 35 to 45 cm. note that this distance depends on the area to be operated on of the patient, and the type of table. However, given the rather uniform design of most tables, the latter will not create discernable variations, while the former is typically linked to the type of table used as well (for instance, Hana® with anterior approach hip surgery).

The advantage of providing such a sleevelike or pocketlike portion/through-hole at the desired position, allows a medical practitioner to maneuver the mounting element (and the instrument mounted thereon) during (or before and after) the surgery, without potentially ripping or dislodging the rest of the surgical drape.

In general, said mounting elements are maneuverable in a vertical position, which perfectly suits the sleeve- or pocketlike form of the portion/through-hole, as this accommodates such movement. However, by introducing a specific portion to receive the mounting element at a predefined distance from the area to be operated on, it can be ensured that there is sufficient leeway in horizontal movements as well (as opposed to simply clamping a random part of the sheet and discovering mid-surgery that it does not provide enough leeway to maneuver).

In what follows, the “sleevelike or pocketlike portion” will often, for the sake of brevity and conciseness, be referred to as “sleeve” or “pocket” without a distinction being made to their form, dimension or structure unless stated otherwise. Furthermore, most advantages discussed hereafter are in fact applicable to the through-hole, though the term “sleeve” is used in the explanation. Nevertheless, the person skilled in the art will readily recognize the advantages that apply to a version of the sheet without a sleeve but only a through-hole instead.

In a preferred embodiment, the sleevelike or pocketlike portion preferably has a diameter comprised between 5 mm and 100 mm, preferably at most 90 mm, more preferably at most 80 mm, even more preferably at most 70 mm, even more preferably at most 60 mm, even more preferably between 15 mm and 58 mm, even more preferably between 25 mm and 55 mm, most preferably between 35 and 50 mm.

In a preferred embodiment of the invention, the sheet is adapted for orthopedic procedures.

In a preferred embodiment, the sheet is adapted for hip surgery. In an alternative embodiment, it is adapted for knee surgery. It should be understood that the adaptation lies amongst others in specific dimensions and orientations of certain features (sleeve, anchoring zones, etc.).

In a preferred embodiment, the sleeve has a length of at least 5 cm, preferably at least 10 cm, more preferably at least 12.5 cm or even 15 cm (i.e. capable of receiving an elongate object with a length of 5 cm, 10 cm, 12.5 cm and 15 cm respectively entirely within the sleeve).

In a preferred embodiment, the sleeve has a length of at most 60 cm, preferably at most 50 cm, more preferably at most 40 cm, even more preferably at most 30 cm, or even 25 cm or 20 cm.

In a preferred embodiment of the invention, said sleevelike or pocketlike portion is adapted to receive a first elongate element from the proximal surface, and whereby the sleevelike or pocketlike portion comprises a material suitable for allowing a second element to embrace the distal end of the first elongate element from the distal surface.

Preferably, the sleevelike or pocketlike portion comprises a transparent or translucent material.

It is of tantamount importance that the sleeve is adapted to withstand being clamped between two ‘connecting’ elements, an instrument holder and an instrument. Although this connection is typically not fastened by an actual clamping element, but rather an ‘embracing’ connection via a fitting male and female connection which is ensured by the weight of the inserted element, the material of the sleeve still needs some strength as the instrument holder may be moved by a surgeon or assisting personnel, which can create stress on the sleeve. Additionally, friction from the instrument turning with respect to the instrument holder can create additional strain and tear the material. Therefore, it is important that the material is somewhat tear-resistant, for instance comprising latex. The material of the sleeve may comprise a plurality of layers, with similar and/or different properties (liquid resistant, absorbent, transparent, tear resistant, etc.).

In a particularly preferred embodiment, the sheet comprises at least one anchoring zone at a third, internal position on the proximal surface of the sheet adapted for positioning, preferably affixing, said anchoring zone on the proximal surface to a biometric anchoring point or zone on the body of the surgical patient, preferably to or around the knee or patella, whereby said third position is spatially separated from the first position over a predetermined distance, said predetermined distance between the first and third position being dependent on the biometric anchoring point or zone, and whereby said third position is spatially separated from the second position over a predetermined distance, preferably whereby the sheet at least partially transparent, translucent or open at or around the anchoring zone.

The applicant notes that by providing a further anchoring zone, outside of the preferred fixation of the incision window at/around the area on the body to be operated on, it is possible to both secure the surgical drape better (as it can no longer be rotate, and as such the sleeve or through-hole will be aligned with the position of the mounting element of the table), and more importantly, to position the surgical drape over the patient in a more controller, more accurate and more sterile fashion. The method of placing the surgical drape over the body of a patient will be discussed further in this document. However, it is clear from the above embodiment that by using a biometric anchoring point (with a reliable expected position for the average patient), the accurate positioning of the incision window can be ensured. Applicant found the patella to be most reliable anchoring point, given the relatively central position of the patella in the lower body of the patient, and especially with a reliable forecast of the distance of patella to the hips (for anterior approach hip surgery). Instead of directly attempting to secure the surgical drape at the area to be operated on, it was found advantageous to first secure at an area of lesser importance (where breach of sterility for instance is less dangerous), from which the correct position of the incision window then follows. Should the position at the biometric anchoring point be incorrect, it can be corrected, and the incision window will be correctly placed. In prior art methods and systems, the incision window is placed first, and its incorrect positioning cannot be corrected without potentially endangering patient, disturbing the sensitive area to be operated on and/or breaching sterility.

It is therefore even more advantageous to provide a (at least partly) transparent/translucent/open fixation window as well, which can be used to confirm the correct position of the fixation window.

In a further preferred embodiment, the sheet comprises at least one adhesive surface at and/or near, preferably substantially entirely around, the at least one anchoring zone, whereby said adhesive surface is provided with a removable cover which covers the adhesive portion.

Thus, in a particularly preferred embodiment, the anchoring zone comprises a central transparent/translucent or open window, which can be aligned with the predetermined biometric anchoring point, around which central window an adhesive layer is provided on the proximal side of the sheet, preferably with a removable cover over said adhesive layer. The shape of the central window is typically circular or oval, but can have other shapes.

In a further preferred embodiment, the anchoring zone is provided to the sheet comprised in a foldable flap, which can be folded against the sheet, and away therefrom via an attached edge or zone of the flap. Preferably, said attached edge is provided on the opposite side of said foldable flap with respect to the through-hole. In case of a symmetric sheet with two foldable flaps, the attached edges are provided on the mutually proximal sides of the foldable flaps with respect to each other. The anchoring zone preferably comprises an adhesive surface on one surface of the flap at, near or around the anchoring zone. The surface of the flap upon which the adhesive surface is provided is that proximal to the patient.

In a further preferred embodiment, the predetermined distance between the first and third position is comprised between 25 cm and 65 cm, preferably between 30 cm and 60 cm, more preferably between 35 cm and 55, and most preferably between 40 cm and 50 cm. The applicant notes that this particular distance is ideally suited for the Hana® table and anterior approach hip surgery. The mounting element of the table has a maximal distance of about 45 cm of the position of the hip on the patient when he or she is correctly positioned on the table (note that due to the construction of the Hana® table, each patient is always positioned at the same place to ensure uniformity).

In a further preferred embodiment, the second position is not substantially aligned with a line defined by the first and third position, and preferably distanced at least 20 cm, more preferably at least 25 cm, from said line. In a particularly preferred embodiment, the distance is comprised between 25 cm and 50 cm, preferably between 30 cm and 45 cm.

The above creates a triangular constellation of the three positions, each of which serve as separate anchoring points: the first and optionally the third are affixed to and anchored on the body of the patient, the second is clamped or clinched at the instrument holder/mounting element. Such a triangular setup further aids in their correct positioning, but also lowers the chance that the drape is shifted, as opposed to a situation where all three are aligned. It is of further note that the mentioned distance relates to the distance of a mounting element on the Hana® table to a line along the length of the leg most proximal to said mounting element.

In a preferred embodiment, the sheet, not considering transparent, translucent or open portions (and potentially the sleevelike or pocketlike portion), substantially comprises three layers, at least one of said layers, preferably at least the layer at the proximal surface, being waterproof, at least one of said layers, preferably at least the layer at the distal surface, being liquid-absorbent, and at least one of said layers, preferably at least the middle layer and optionally the layer at the distal surface, being reinforcing.

As mentioned, certain parts of the sheet will require a specific make-up (transparent section, cuttable section, strengthened, flexible, etc.). However, at least a central part of the sheet substantially comprises said three layers, which ensure optimal operating conditions, namely the guarantee on sterility (reinforced, water-repellent/waterproof) and reducing the prevalence of fluid waste (absorbent). Furthermore, using separate layers increases the strength of the thusly created laminate. It is of further note that said three-layer zone can be used to cover the patient's body, while a fringe zone surrounding the central zone may only comprise one (or two layers), as these outer zones are not as exposed to fluids and other potentially contaminating substances.

In a preferred embodiment, the sleevelike or pocketlike portion comprises a transparent or translucent material.

In a preferred embodiment, the surgical sheet is essentially rectangular. Preferably, the sheet comprises at least one, more preferably two with respect to a central symmetry axis in the sheet oppositely positioned, side flap which extends from a side of the sheet, which side flap is positioned and suitable for covering a limb of the surgical patient. By providing said side flap (or flaps), the sheet is ideally adapted to cover specific extremities of the patient's body, such as his or her limbs, specifically arms. Usually, the arms are held stretched from the shoulders, and thus would not be covered unless the sheet is made overly big. By providing flaps, the sheet itself can be kept to reasonable dimensions.

In a preferred embodiment, the surgical sheet is essentially rectangular, and comprises a central sheet portion wherein the incision window and sleevelike or pocketlike portions/through-hole are positioned, said central sheet portion, not considering transparent, translucent or open portions (and optionally the sleevelike or pocketlike portion), substantially comprising at least two layers, preferably three layers as discussed previously, and whereby the sheet furthermore comprises a fringe sheet portion around the central sheet portion which comprises less layers than the central sheet portion. A fringe sheet portion with less layers will be less bulky, cheaper and more efficient, as there is less chance of blood spatters and other contaminants further from the area to be operated on. Furthermore, a rectangular lay-out allows easier folding and unfolding.

In a preferred embodiment, the surgical drape is suitable for use in hip surgeries, preferably whereby the distance between the incision window and the anchoring zone is suitable for positioning, and preferably thereby affixing, the anchoring zone to the patella or knee of a patient. It is typical for hip surgery operations to have a movable mounting element for instruments on the side(s) of the table (for instance, Hana®), as such, the surgical drape can be easily made (dimensioned, especially inter-position distances) to be perfect for almost any patient undergoing hip surgery (unless massively over- or undershooting the average body dimensions of a human).

In a preferred embodiment, the sheet comprises at least one adhesive surface at and/or near, preferably substantially entirely around, the at least one incision window, whereby said adhesive surface of the incision window is provided with a removable cover which covers the adhesive portion of the incision window. Note that said adhesive surface is positioned on the proximal side of the sheet. The advantages of an adhesive region around the incision window are self-evident, namely the possibility to secure the incision window around the area to be operated on.

In a particularly preferred embodiment, the sheet is mirror symmetric with respect to a central line along the length of the sheet, each symmetric side comprising at least one of said transparent or open incision windows, and at least one, preferably one, of the through-hole (and preferably said through-hole being provided with, preferably closed, sleevelike or pocketlike portions as discussed previously), and preferably whereby each symmetric side comprises at least one of said anchoring zones as disclosed earlier.

The advantages of a symmetric sheet is that each sheet can be used for surgery on both sides of the body (so there is no danger that the wrong sheet is picked for a surgery), as well as being especially fit for double-sided surgeries (which occur often for anterior approach hip surgeries).

Additionally, it allows for a further securing of the sheet, via the incision window on the opposite side, and optionally the sleeve/through-hole (and/or the anchoring zone to the biometric anchoring point on the other side of the body of the patient).

In a preferred embodiment, the sheet is at least 200 cm long, preferably at least 300 cm, more preferably about 400 cm long, to ensure full coverage of the patient, and allowing the sheet to hang over the entire table. In a preferred embodiment, the sheet is at least 75 cm wide, preferably at least 90 cm wide, more preferably at least 105 cm wide, and most preferably about 115 cm wide, again to ensure full coverage.

In a preferred embodiment, the incision window is substantially trapezoid, preferably right, whereby the shorter base edge is towards the head side of the sheet (away from a potential anchoring zone). In some embodiments, the surrounding adhesive surface is rectangular and share one, two or three parallel sides with the trapezoid incision window. Alternatively, it may be trapezoid as well. The incision window preferably has a surface of at least about 300 cm², more preferably at least 450 cm², and even more preferably at least 600 cm². The diameter of the incision preferably is longest along an axis from the head side to the feet side of the sheet.

In a preferred embodiment, the anchoring zone comprises an essentially circular or oval transparent portion, with a diameter of at least 8 cm, preferably at least 10 cm, more preferably between about 12 cm and 18 cm. Around said transparent portion, an adhesive surface is provided over at least 2.5 cm from the edges of the transparent portion. The adhesive surface preferably is provided in the form of a rectangle.

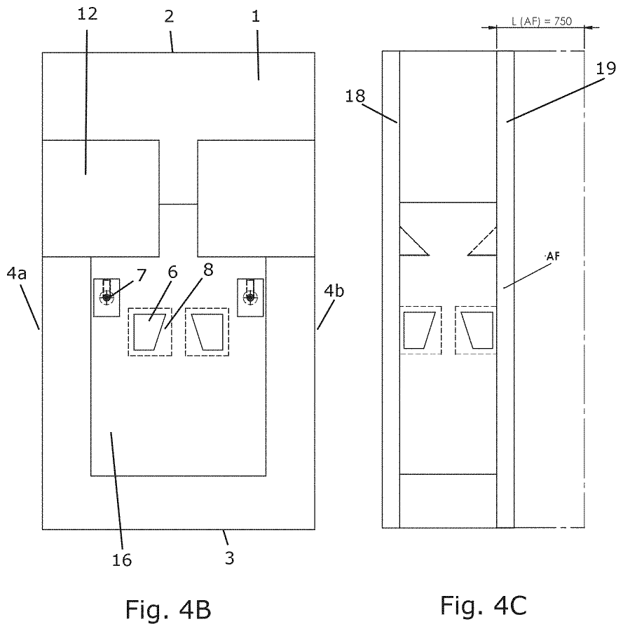

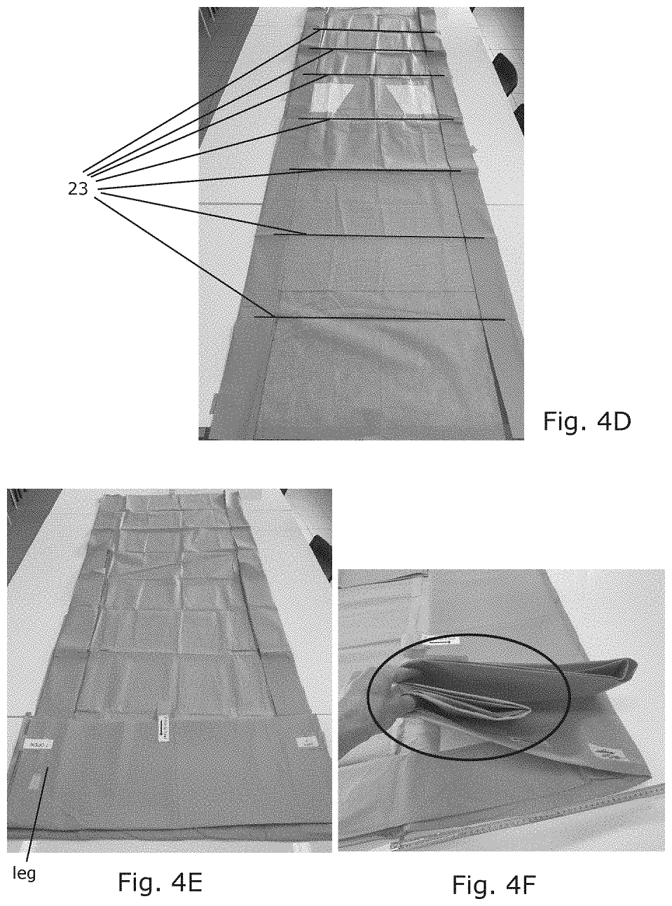

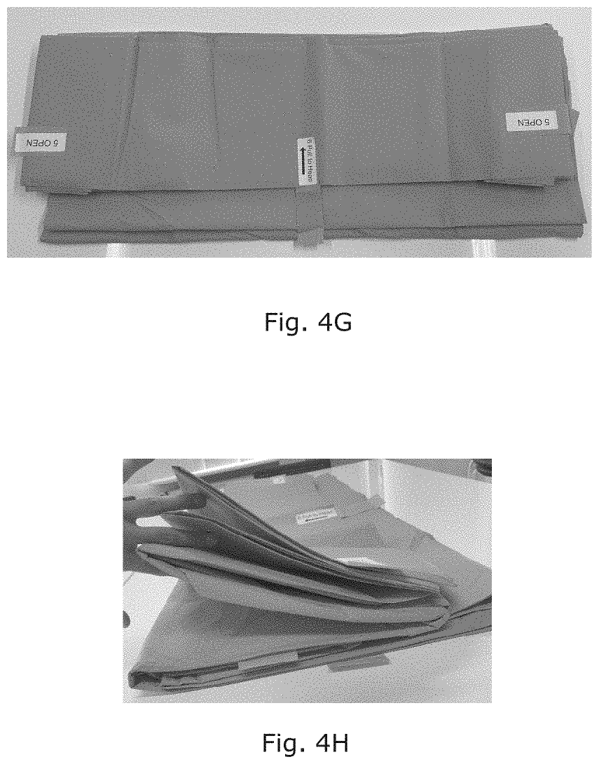

In a preferred embodiment, the drape is provided with cover sheets at the incision windows, said cover sheet essentially covering the entire surface of the folded surgical drape (as seen in an embodiment in FIG. 4A), when folded according to the methods described in this document.

In a second aspect as discussed previously, the invention a method for unfolding and positioning a folded surgical drape according to the present invention, on a body of a surgical patient which is located on an operating table, said table comprising at least one lateral, movable, substantially vertically extending, mounting element for receiving modular surgery instruments.

Again, the advantages are apparent.

The applicant has specifically adapted the surgical drape, as well as the method of folding said surgical drape, to allow an extremely practical method of unfolding it and positioning it over a patient's body on an operating table. During the unfolding and positioning, it is crucial that sterile areas are shielded from outside interference, which means that the way the drape unfolds needs to happen in a very specific order, and with specific orientations (for instance, a surface that touched a non-sterile zone may not be unfolded to ultimately or intermediately touching a sterile zone). Additionally, the structure of the drape and its components allow it to be positioned correctly with respect to the area to be operated on, and a mounting element of the table, so the respective incision window and sleeve/through-hole are at the correct place when further unfolding the sheet.

In a first step (optionally preceded by others, as will be discussed further on), the sheet is placed so that the incision window is positioned over the area of the body to be operated on, allowing the surgeon full view of said area, whereby the adhesive portion around the incision window is affixed to the skin of the patient at or around the area to be operated on. As mentioned, the incision window usually is a transparent film, which is cut during surgery to allow access to said area. It is important to perform this step early on in the deployment of the sheet, as an incorrect positioning in this step, will necessitate the removal of (almost) the entire sheet, to be reapplied, or replaced by a new sheet. As such, it is desirable to ascertain the correct position of the incision window as early as possible.

Once the incision window is correctly positioned, the sheet may optionally (preferably) be further unfolded so that the sleeve/through-hole is made free so it can be placed over the mounting element to the side of the operating table. Usually, this will require the sheet to be unfolded further laterally so the sleeve/through-hole becomes accessible from both surfaces. Once this is completed, a modular instrument may already be positioned over the mounting element, thereby potentially clamping the sleeve between the instrument and the mounting element. However, this step may also be performed later on, before, during and after the surgery, and can be repeated a number of times throughout. Once the incision window and the sleeve/through-hole are positioned correctly, the sheet can be fully unfolded, covering essentially the entire body of the patient. Note that in some embodiments, the sheet is only dimensioned to cover part of the body, for instance not the feet and/or the head (or even not the upper torso), and/or the sheet may be adapted to only cover one lateral side of the body. Preferably however, the sheet is dimensioned to cover the entire body.

In a preferred embodiment, and with a surgical drape comprising one or more anchoring zones as disclosed earlier, the method comprises a step of positioning, and preferably affixing, the anchoring zone to a biometric anchoring point or zone on the body of the patient, whereby the step of positioning, and preferably affixing, the anchoring zone to the biometric anchoring point or zone precedes the step of positioning the incision window over the area to be operated on.

The applicant noticed that in order to correctly position the incision window, it was highly advantageous to be provided with an anchoring point of the sheet to the body that is in place, before positioning the incision window on the body of the patient. As the sheet is still at least partly folded at this point (to allow convenient handling, as opposed to a fully unfolded sheet which is too large to handle properly), the incision window(s) is usually not visible from the distal side of the sheet where the surgeon or surgical assistant is standing who applies the sheet. In said position, the fact that they cannot see the incision window is inconvenient as they cannot ascertain whether the incision window is positioned correctly on the area to be operated on. It is no solution to only apply the incision window at a later phase, when the sheet is almost fully unfolded, since at that point, it is no longer possible to apply corrections if the incision window is not lined up correctly with the area to be operated on, as this could breach sterility, which means the entire sheet would need to be removed, and a new sheet would need to be placed (which process is subject to the same problems as before).

It is in this light that the applicant adapted the surgical drape to comprise one or more biometric anchoring points or zones (preferably two, symmetrical for full-body drapes). These anchoring points allow the staff to position (and optionally affix) the sheet with respect to a ‘safe’ part of the body of the patient (with no real danger to sterility), which point can be used to almost guarantee the correct positioning of the incision window. By this adaptation, the person unfolding no longer needs to see through the incision window to know that it is positioned correctly, as it follows from the correct positioning of the anchoring zone.

We note that the suggested approach could only fail if the expected distance between the anchoring point and the incision window would be gravely different from an average value, and given that the incision window is already oversized to allow some variation to work for 99% of all patients, it can easily be further enlarged to work for all but 100% of the patients. Additionally, trained staff can easily identify such diverging situations and take other measures (for instance, using variations off the sheet which are adapted for such over- or undershoots of the expected distance). By deliberately dimensioning the sheet, and specifically the distances between the anchoring zone and the incision window (and by default also the sleeve/through-hole), the proposed sheet and associated unfolding method guarantee a safe (sterile), easy and accurate position of the unfolded sheet with respect to the body of the patient and with respect to the operating table.

In an especially preferred embodiment, the anchoring zone is first positioned, and preferably affixed, after which the sheet is unfolded whereby the incision window automatically ends up at the correct position. This is notably an extremely reliable method in an embodiment where the sheet is symmetrical to cover both sides of the body of a patient, and where two anchoring points can be used, resulting in an automatically correct alignment to the incision window (where only the distance between anchoring point and area to be operated on can vary slightly, but will normally fall within the range the incision window is dimensioned to account for). In this case, the sheet may be presented ready to be positioned, and preferably affixed, to both anchoring points at once, or can be first positioned (and optionally affixed) to one, and then unfolded to be positioned (and optionally affix) to the other (as per the Figures, for instance). The latter is again advantageous as the unfolding automatically places the anchoring zone at the correct location, while the folded sheet is kept extremely compact. Note that in some variations, the two biometric anchoring points are the same (such as using the navel).

In a preferred embodiment, the biometric anchoring point or zone is the patella and/or knee of the patient on the same lateral side of the body as the area to be operated on.

The applicant noticed that the patella is an especially reliable anchoring point, as it has a very distinct position with respect to other body parts commonly operated on (for instance hip), as well as being highly ‘recognizable’, both visually as via tactile sensations or touch. The distance, as well as the orientation, in view of the patella, can be easily estimated and does not vary tremendously (in absolute distance), as the patella is relatively centrally positioned, while still allowing enough distance to avoid sterility issues. Typically, a member of the surgery team may provide a marker on the patella, which allows the anchoring zone to be aligned more easily when said anchoring zone comprises a window which is see-through (transparent, translucent, or even open).

A specific embodiment of the unfolding method will be discussed under examples, but it is of note that some unfolding steps are not necessary, but preferably present, as they can make the (partially) folded sheet more manageable for deployment. However, the core of the unfolding method lies in the fact that the sleeve/through-hole is folded over the mounting element after the incision window is affixed. A further inventive aspect in the method is that between affixing the incision window and placing the sleeve/through-hole over the mounting element, the portion of the sheet comprising the sleeve/through-hole is unfolded laterally (to ‘free’ up the sleeve/through-hole). In another inventive aspect, the use of an anchoring zone to first position, and preferably affix, the sheet, after which the incision window is affixed, greatly improves correct and sterile positioning. In this aspect as well, it is preferred that after positioning the anchoring zone, the portion of the sheet comprising the incision window is unfolded from the attached portion, guaranteeing sterility, as well as the automatic correct positioning. After having secured at least the incision window(s) and the sleeve(s)/through-holes (and preferably the anchoring zone(s) as well), the sheet can then be finally fully unfolded, for instance, by unfolding a laterally extending portion suitable for covering limbs of the patient.

In a third aspect as discussed previously, the invention a method for unfolding and positioning a folded surgical drape according to the present invention, whereby the surgical drape is a symmetrical surgical drape as disclosed, on a body of a surgical patient which is located on an operating table, said table comprising at least one lateral, movable, substantially vertically extending, mounting element for receiving modular surgery instruments. As mentioned previously, a symmetrical surgical drape is often used, as the cost is not much higher than a ‘single-side’ surgical drape, while it is more versatile, and can be fixated more easily and more accuracy. Furthermore, the applicant notes that in some surgeries, for instance hip surgeries, the operation is often performed on both sides of the body in a single procedure, which provides further advantages to the symmetrical sheet, aside from the additional fastening to the body of the patient.

In a preferred embodiment, with a surgical drape comprising anchoring zones on both symmetrical lateral sides, the method comprises following steps:
positioning, preferably affixing, a first of said anchoring zones of the sheet to the biometric anchoring point or zone on a first lateral side of the body of the patient;
positioning, preferably affixing, a second of said anchoring zones to the biometric anchoring point or zone on the second lateral side of the body of the patient;

whereby the steps of positioning, and preferably affixing, the anchoring zones to the biometric anchoring points or zones precede the steps of positioning the incision window over the area to be operated on.

By first fixing both of the anchoring zones on the (preferably symmetric) anchoring points, for instance patella), the position of the incision window is ensured to align with the actual area to be operated on, as having two anchoring zones necessarily ‘forces’ said alignment. Again, by having these steps precede others, mistakes can still be corrected without endangering the sterility of the operation. In a most preferred embodiment, the anchoring zones of the sheet allow a user to see through (if not at the time of fixation, then at least before positioning, and optionally affixing, the incision window) to the distal side so they can ascertain the correct alignment to the anchoring points. As noted, a mark may also be applied to the anchoring point by the operating team, so the alignment can be more easily, visually verified.

Preferably, the step of positioning, and preferably affixing, the first of said anchoring zones to the body of the patient, is preceded by the following step: unfolding a flap comprising the first anchoring zone from the folded sheet, to overlay (with said flap, and specifically the first anchoring zone) the biometric anchoring point or zone on the first lateral side of the body of the patient.

Preferably, the step of positioning and preferably affixing the second of said anchoring zones to the body of the patient, is preceded by the following step: unfolding a flap comprising the second anchoring zone from the folded sheet, to overlay (with said flap, and specifically the second anchoring zone) the biometric anchoring point or zone on the second lateral side of the body of the patient.

Again, in certain embodiments the first and second biometric anchoring points may coincide, such as when the navel is used as biometric anchoring point.

In a fourth aspect, the present invention provides a method for folding up a surgical drape according to the invention, said sheet being essentially rectangular and having a head side, a leg side, and two lateral sides, with the method as detailed in the claims. Note that the preferred options need not be performed as an all-or-nothing package. Furthermore, not that the relative steps of

In a first stage, the surgical drape is fully unfolded, and thus in an essentially flat, rectangular shape, with a sleeve/through-hole and an incision window in the sheet, on predefined distances (and orientations with respect to each other and the general form of the sheet). Note that in a preferred embodiment, the sheet is symmetrical, with two of the aforementioned elements on each symmetrical side. In one of the first steps, the lateral section with the sleeve/through-hole is folded over the rest of the sheet. In case of symmetry, both lateral section are folded over the central zone. Preferably, this results in a more narrow rectangular form (though parallelogram/trapezoid shapes are possible as well, but less preferred). Note that the folded version does not cover the incision window. By allowing the incision window to be uncovered even when the lateral section with the sleeves/through-holes are folded, this enables the user to ascertain proper alignment with the area to be operated during deployment.

In another first step (may be switched in the order with the previously mentioned step of folding the lateral regions), the head side section is folded over to at least partly cover the incision window. Preferably however, the head side section is folded to only partly cover said incision window at most, which allows the user to already see the alignment with the area to be operated on while the head side section is still folded (partly) over the incision window.

Note that one or more, preferably all, of the above folds are preferably accordion folds, which allow the incision window to be kept free, while greatly reducing the width and length of the sheet. A few (non-limitative) examples of the accordion folds are shown in the Figures as well.

In a preferred embodiment, the head side section of the already folded sheet, is then further folded over the rest of the sheet, said further folded section comprising the incision window(s). Note that this is especially useful when the sheet comprises one or more anchoring zones, which lie in the section of the sheet covered by the further folded section, as this would ensure that unfolding the further folded section during deployment, means that the incision window is folded towards its correct position, covering the area to be operated on, which is guaranteed as the anchoring zones ensure the proper positioning.

Furthermore, the leg side section may be folded over the remaining sheet to further reduce the size.

In a variation, the method of folding comprises the following steps:
a. folding up a section of the sheet on the lateral side of said sheet most proximal to the through-hole, over the sheet substantially up to, but not over, the incision window, and whereby said folded section preferably comprises said through-hole and the associated sleevelike or pocketlike portion, more preferably whereby the folded section on the lateral side is accordion folded;
b. folding up a section of the sheet on the leg side, preferably at least partly, more preferably at most partly or alternatively entirely, over the incision window, preferably whereby the folded section on the leg side is accordion folded, more preferably whereby the folded section on the leg side is doubly accordion folded;
c. folding up the section of the sheet on the head side which is not folded over with the folded leg side section, preferably whereby the folded section on the head side is accordion folded, more preferably whereby the folded section on the head side is at least doubly, even more preferably at least triply or quadruply accordion folded.

Note that the person skilled in the art would readily understand that most, if not all of the further embodiments may be applied to both the first method of the fourth aspect, as well as to the variation described above, and as such form part of the present disclosure.

As mentioned previously, in a preferred embodiment, wherein the surgical drape comprises one or more anchoring zones as disclosed earlier, the further folded section covers the anchoring zone(s). It should be understood that, depending on the point of view, the further folded section is folded to cover the anchoring zone(s), or the section comprising the anchoring zone(s) could also be considered as folded to cover the further folded section comprising the incision window(s).

In a preferred embodiment, wherein the surgical drape is symmetrical as disclosed earlier, the method comprises a final folding step of folding the sheet over the central symmetry axis onto itself.

In a preferred embodiment, wherein the surgical drape comprises one or more side flaps, the method comprises the step of folding the one or more side flaps over the sheet previous to further folding actions.

In a preferred embodiment, one or more folding steps is followed by a step of providing one or more seals attaching the folded sections to another part of the sheet. It is important that the folded sheet cannot accidentally unfold at inopportune times, so the folded sections are sealed to the rest of the sheet, thereby only allowing the folds to be unfolded by a deliberate removal of the seal(s).

Lastly, the invention pertains to the use of a surgical drape according to the embodiments discussed in this document, during a surgical procedure, specifically in hip surgery, more specifically in anterior approach hip surgery.

The invention is further described by the following non-limiting examples which further illustrate the invention, and are not intended to, nor should they be interpreted to, limit the scope of the invention.

The present invention will be now described in more details, referring to examples that are not limitative.

### EXAMPLES

### Example 1: Folding Up a (Full-Body) Surgical Drape

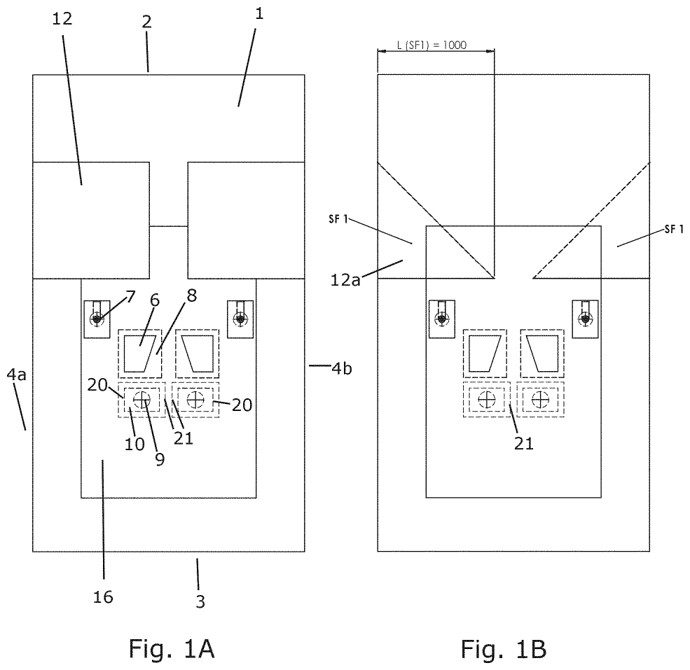

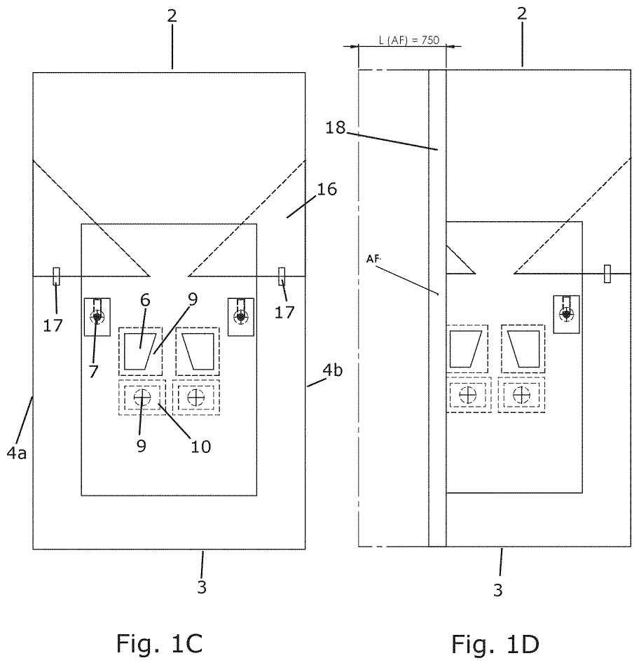

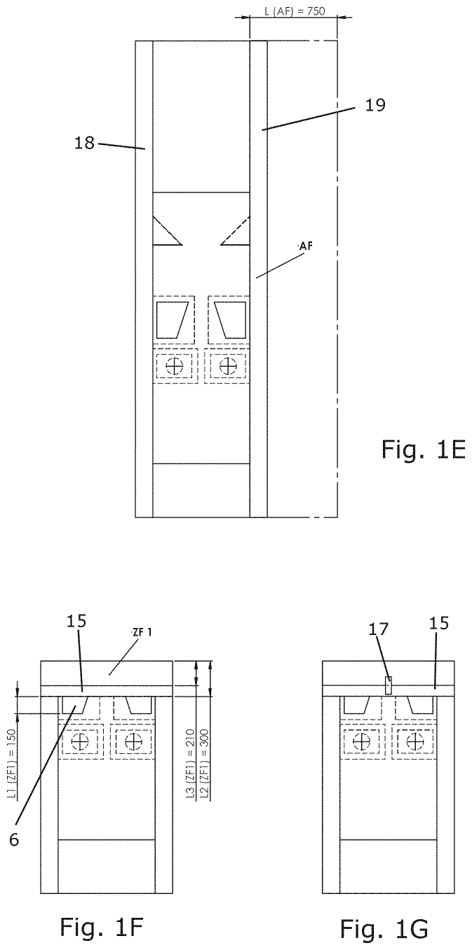

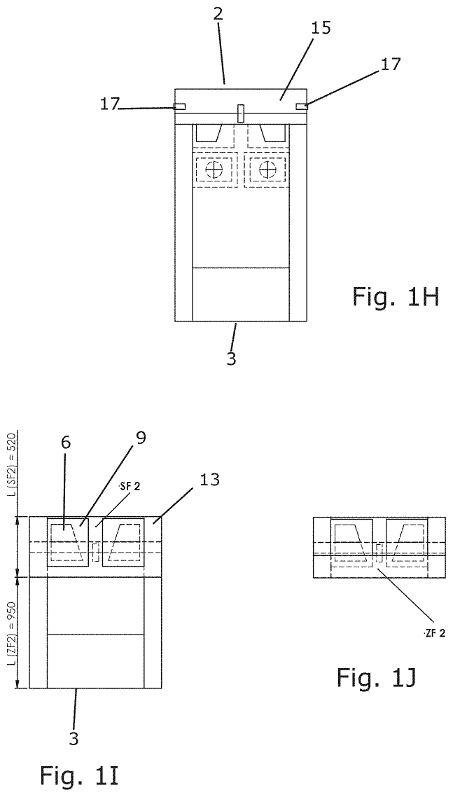

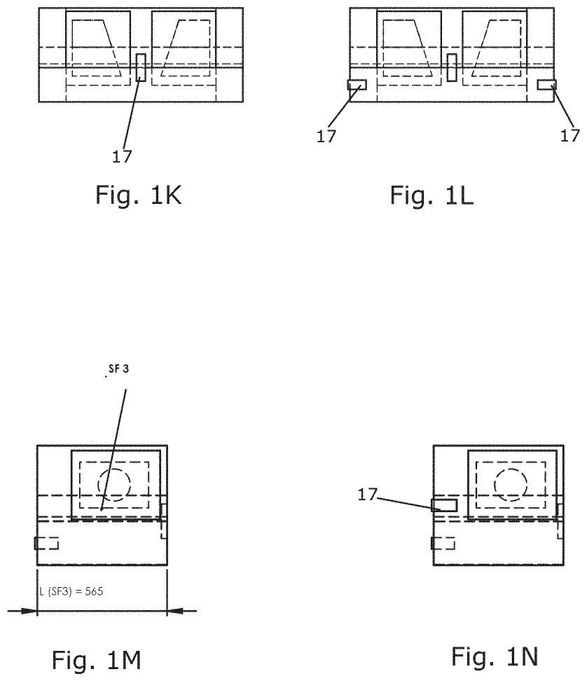

FIG. 1A shows the unfolded surgical drape (1), where the limb-covering side flaps (12) are already folded inward over the ‘body’ of the sheet. Note that a central portion (16) of the sheet is visible, which can be of a different material make-up than the rest (typically, more layers).

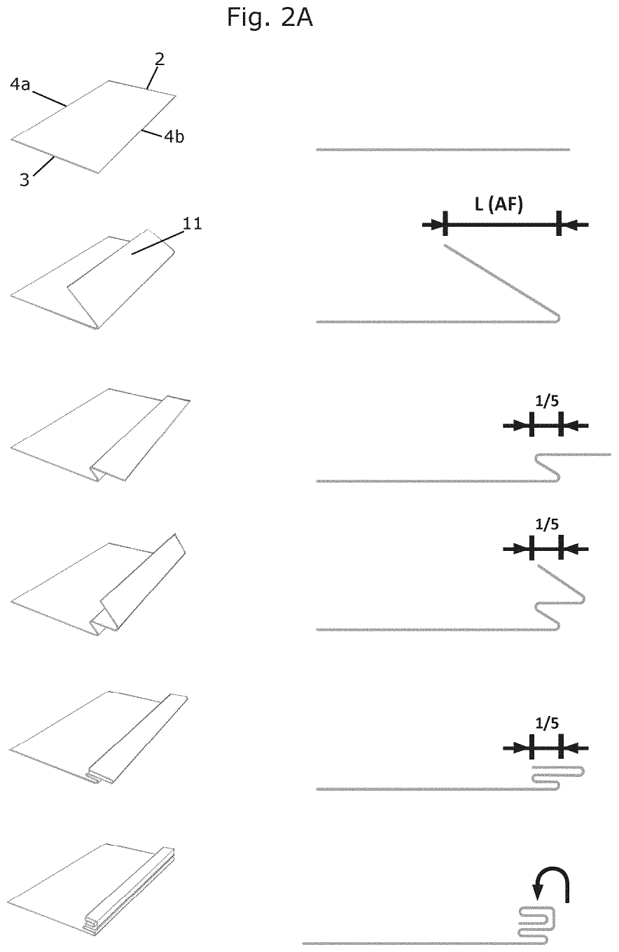

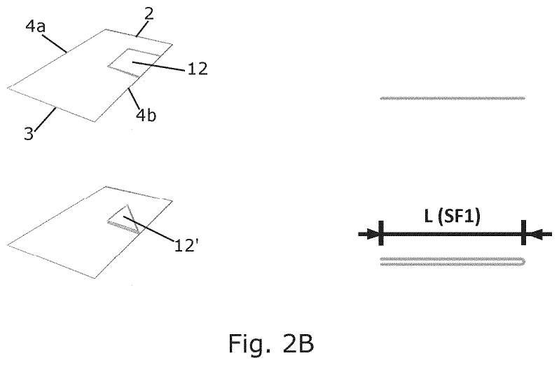

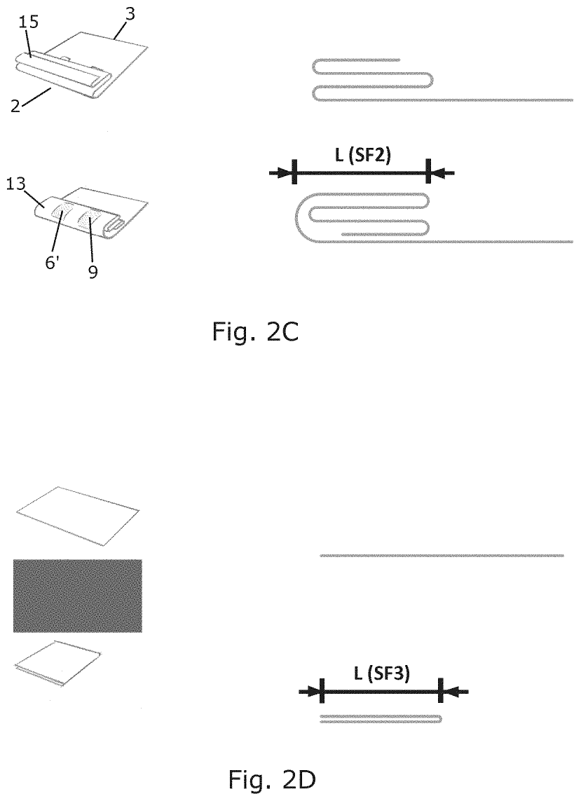

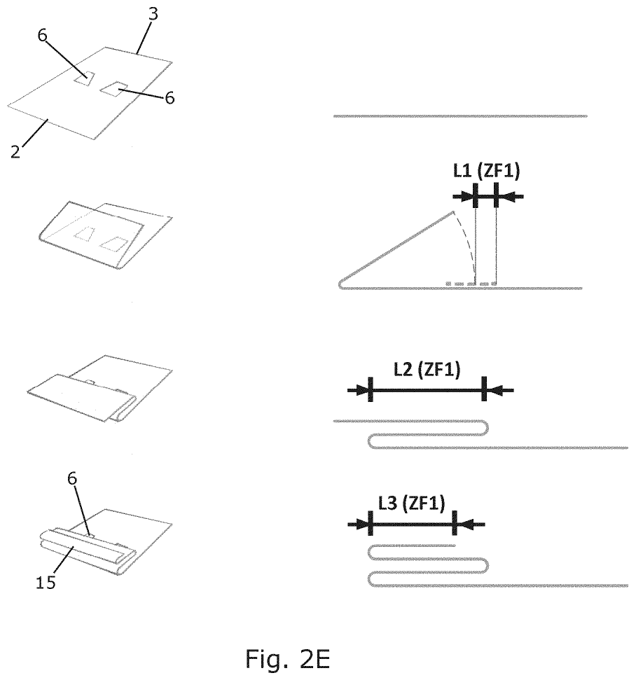

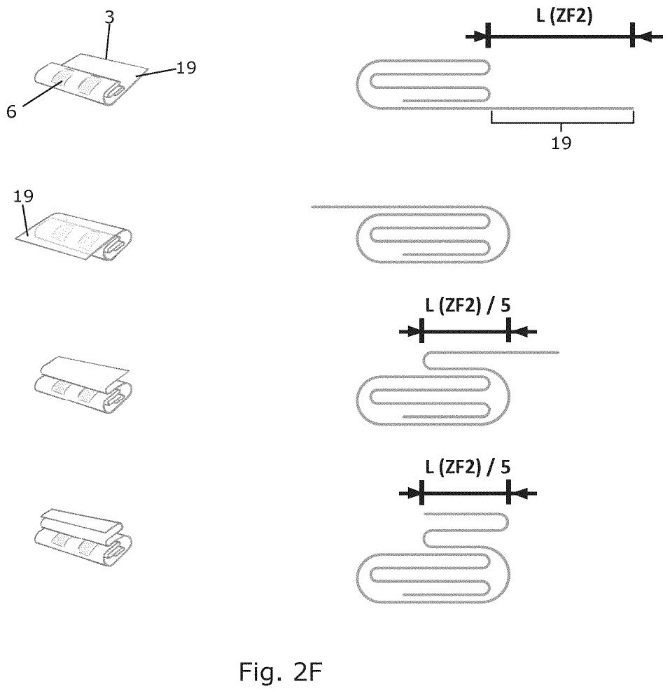

In a possible first step, the side flaps (12) may be folded another time onto themselves, as can be seen in FIG. 1B and FIG. 2B. Note that preferably, seals (17) are provided to secure the folded side flaps (12′), as seen in FIG. 1C.

In a further step, as can be seen in FIG. 1C-1D, a lateral section (11) on a side (4a) of the sheet, is folded (accordion folded as seen in FIG. 2B), up to the incision window (6, 9), but not covering it. Said folded lateral section (18) comprises the sleeve (7). The accordion fold can count 2, 3, 4 or more separate plies, and it is to be understood that the main objective is to reduce the dimensions of the folded sheet as much as possible. On the other lateral side (4b), the fold (11) is also performed, resulting in the partly folded sheet of FIG. 1E. In the next step, a section on the head side (2) of the sheet is folded towards the center of the sheet as seen in FIG. 1F, again preferably in an accordion fold (for instance as seen in FIG. 2C). Note that the folded head section (15) does not cover the incision windows (6) entirely. Again, a seal (17) is applied to fasten the fold (15), this time onto itself (FIG. 1G), as well as to the rest of the sheet (FIG. 1H).

In a following step, the section on the head side (2) of the sheet is further folded. In particular, the head section is further folded so that the incision window (6) is comprised in the further folded section (13), said fold with a relatively low margin beyond said incision window (6), preferably at most 20 cm, but more preferably at most 10 cm, or even 5 cm. In the most preferred version, as per FIG. 1I and FIG. 2E, the further folded section (13) is folded with the fold line at the edge of the adhesive surfaces (9) around the incision windows (6). Note that at the end of this step, the incision windows (6) will be exposed on the top side of the partly folded sheet, while the anchoring zones (8) are exposed on the bottom side of partly the folded sheet (or the other way around depending on how it was folded), which allows the deployment as discussed.

In a following step, depicted in FIG. 13, the section on the leg side (3) of the sheet is folded over the rest of the sheet, again in an accordion fold. First the remaining (unfolded) leg section (19) is entirely folded over the rest of the sheet, after which it is doubled back over itself twice (see FIG. 2F). A number of seals (17) are then applied (FIG. 1K-1L). Note that the anchoring zones (8) still are exposed at this point.

In the final step, the sheet is folded over its vertical axis (symmetry axis) (5) to further reduce size, and provided with a seal (17) in FIGS. 1M and 1N, and FIG. 2D, with the anchoring zones (8) being exposed at the front and back side of the fully folded sheet.

Note that in the examples, the sheets are dimensioned for hip access via the incision window and fixation at the patellae. However, based on the present disclosure, the concept can be easily adapted for other surgeries and/or fixation points by the person skilled in the art.

### Example 2: Unfolding a Full-Body Surgical Drape

Departing from the fully folded sheet from above, which is typically placed in an airtight bag, the user finds the two fixation zones (8) exposed. The user thus positions the bottom fixation zone (8), the one exposed on the back of the fully folded sheet, and positions this on the biometric anchoring point on the matching side of the body. The user then unfolds the sheet from the anchored side thereof, so the free fixation zone (8) faces downwards, after which it can be affixed to the other biometric anchoring point on the other side of the body. Typically, a cover is removed from an adhesive surface (10) at and/or around the fixation zone (8) before fixation.

A leg section is subsequently unfolded from the sheet, towards the feet of the patient's body. Note that this step is not necessarily present, as in some embodiments, it is not necessary that the sheet unfolds further towards the feet of the patient (depending on the area to be operated on, and on the anchoring point). Preferably however, this step is performed, as full coverage of the entire body is usually recommended.

Subsequently, the sheet is unfolded upwards, whereby the incision windows (6) are automatically folded towards the area to be operated on, of the patient, as is ensured by the preceding correct positioning of the fixation zones (8).

In a further step after affixing the incision windows (6) (again, typically via a peel-away cover over an adhesive surface at and/or around the incision windows), the sheet may be folded upwards even further in order to cover the entire patient.

Subsequently, the sheet is unfolded laterally, thereby freeing up the sleeves (7), which, due to the fixation of the incision windows (6), will be essentially provided at the position of the mounting elements of the table, and can be slid over said mounting elements.

Lastly, side flaps can be unfolded laterally even further, to cover such extremities as limbs (arms).

The above steps can be easily derived from tracing the folding steps of FIGS. 1A-1N and FIGS. 2A-2F backwards.

Note that the removal of any seals (17) is not mentioned, as the seals (17) are not necessarily present, nor is this step challenging enough to warrant mentioning.

In a variation, with a sheet without anchoring or fixation zones (8), the sheet is directly affixed with the incision windows (6) to the zones to be operated on.

### Example 3: Full-Body Surgical Drape

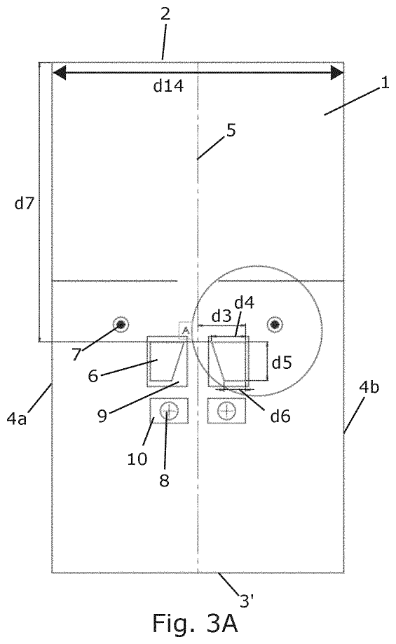

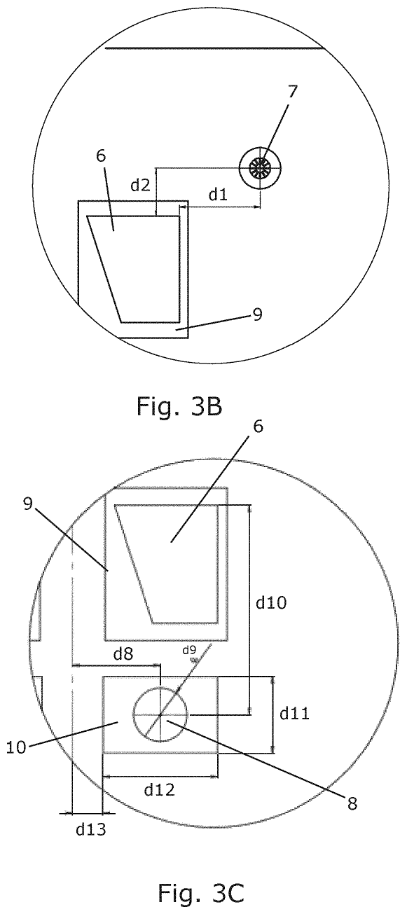

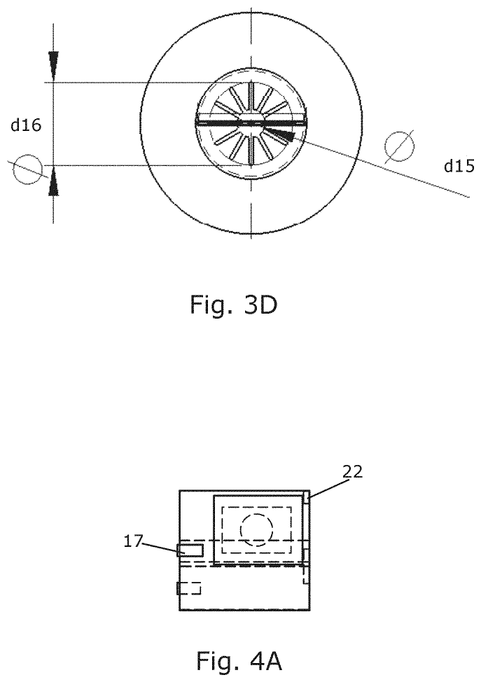

FIGS. 3A-3D show a surgical drape (1) for hip surgery, comprising a head side (2), a leg side (3) and two lateral sides (4a, 4b), said drape fully symmetric over the longitudinal symmetry axis (5). The drape is provided with two incision windows (6) substantially central lengthwise in the sheet, which are each provided with surrounding adhesive surfaces (9) on the proximal (to the patient) part of the sheet. More towards the leg side (3) anchoring zones (8) are provided for each incision window (6), whereby the anchoring zone (8) and the associated incision window (6) form an axis essentially parallel to the symmetry axis.

The anchoring zones (8) are surrounded by adhesive surfaces (10) as well, again on the proximal surface of the sheet.

More towards the lateral sides (4a, 4b) than the incision windows (6), the sheet comprises a sleeve (7) on each side, slightly more towards the head side (2) of the sheet than the incision windows (6).

Additionally, it should be noted that the anchoring zones (8) may be provided in flaps (20) which are attached on one edge (21) to the sheet, and can be folded out from the sheet. This is especially useful when positioning and unfolding the sheet, even more so when dealing with a full body sheet with two anchoring zones (8). In a first step, the folded sheet can be provided essentially vertical to a lying patient, between the legs of said patient (although for single-side embodiments, it can also be provided lateral to both legs). The flap (20) comprising the anchoring zone (8) can then be unfolded (at one or both sides, again depending on the embodiment) to overlay the biometric anchoring point or zone, at the patella in this case. Typically, a cover will need to be removed to free the adhesive zone (10) at or around the anchoring zone (8). Upon fixation, the rest of the sheet can be unfolded further. The main reason to first unfold the flap (20), is that the anchoring zone (8) is preferably transparent (or translucent or open), and thus allows easy positioning on the exact biometric anchoring point.

Furthermore, note that in FIG. 3D, the specific construction of the sleevelike portion is shown, with the through-hole having a diameter d16, from which flexible fingers extend towards the center, up to a smaller opening with a diameter d15. The flexible fingers are adapted to hold back the sleeve somewhat, so it only ‘unrolls’ when intended to, allowing a maximal diameter of the sleeve up to d16. Furthermore, the set-up may be adapted so the through-hole comprises a receiving structure (a ring for instance, as in the FIG. 3D) in which a module sleeve (rolled up) may be introduced and affixed.

In the FIGS. 3A-3D a number of distances are referred to, for which we will provide a range below, as well as a preferred value, in mm.

| | | | More preferred | |
|---|---|---|---|---|
| | Range | Preferred range | range | Preferred value |
| d1 | 150-350 | 180-300 | 220-250 | 235 |
| d2 | 100-180 | 120-160 | 130-150 | 140 |
| d3 | 300-500 | 320-440 | 360-400 | 380 |
| d4 | 200-350 | 240-300 | 260-280 | 270 |
| d5 | 220-400 | 275-350 | 300-320 | 310 |
| d6 | 120-220 | 150-190 | 160-180 | 170 |
| d7 | 1750-2750 | 2000-2500 | 2100-2400 | 2240 |
| d8 | 175-275 | 200-250 | 220-240 | 230 |
| d9 | 100-180 | 120-160 | 130-150 | 140 |
| d10 | 450-650 | 500-600 | 525-575 | 550 |
| d11 | 150-250 | 175-225 | 190-210 | 200 |
| d12 | 200-400 | 250-350 | 280-320 | 300 |
| d13 | 60-100 | 70-90 | 75-85 | 80 |
| d14 | 1750-2750 | 2000-2600 | 2100-2500 | 2340 |
| d15 | 2-30 | 5-25 | 10-20 | 15 |
| d16 | 15-75 | 25-65 | 35-55 | 45 |

### Example 4: Full-Body Surgical Drape with Navel as Biometric Anchoring Point

In a variation, the anchoring zone is used to—without fixation to the patient's body—position the surgical drape with respect to the patient's body. In this embodiment, the navel is used as the biometric anchoring point. An embodiment of this can be seen in FIG. 4A-C. FIG. 4A shows a folded version, where a marker is present on the drape to indicate where the biometric anchoring point (navel in this example) should be positioned. FIG. 4B-C show an unfolded version in various states of unfolding/folding, in which the fixation zones of the previous embodiment are absent, but instead, an anchoring zone (22) may be (visually) indicated, in this case by an appointed area or marker indicating where the drape is to be positioned with respect to the biometric anchoring point.

### Example 4: Folding Up a (Full-Body) Surgical Drape

In a variation, the steps of folding (and thus logically also for unfolding) the surgical drape may vary at certain points. For instance, in an embodiment, steps of FIGS. 1A up to 1E may be followed. However, after this, instead of first folding the head section, as is the case in FIG. 1F and following, the leg section may be folded first, after which the head section can be folded. In this embodiment, an even further reduction of the risk of contamination is achieved.

The method steps which deviate from example 1 are shown in FIGS. 4D, 4E, 4F, 4G and 4H. FIG. 4E shows the step following after that of FIG. 1E, and shows the drape wherein the leg section is folded first. FIG. 4F shows the drape with the folded leg section, and specifically shows the double accordion fold used. FIG. 4G shows the drape with the head section also folded in, with FIG. 4H showing the folded head section, and specifically showing the triple accordion fold used. Note that the fold lines (23) for the accordion folds are already visible in FIG. 4D.

It is supposed that the present invention is not restricted to any form of realization described previously and that some modifications can be added to the presented example of fabrication without reappraisal of the appended claims. For example, the present invention has been described referring to surgical drapes for hip surgery, but it is clear that the invention can be applied to other surgeries as well, for instance stomatological, gynecological, thoracic, cardiac, abdominal, urological, leg surgery, etc.

1-26. (canceled)

27. Surgical drape consisting of a one-piece sheet having a distal surface and a proximal surface relative to a surgical patient, the proximal surface being adjacent to the patient, wherein the sheet comprises at least one transparent or open incision window at a first, internal position, which transparent or open position extends through the sheet, whereby the surgical drape is characterized in that the sheet comprises at least one through-hole through the sheet at a second, internal position, from which through-hole a sleevelike or pocketlike portion extends, said through-hole defining an opening of the sleevelike or pocketlike portion, preferably said sleevelike or pocketlike portion being closed at the extremal end, preferably whereby said through-hole comprises reinforced edges, whereby said through-hole has a diameter comprised between 15 and 75 mm, preferably between 25 and 65 mm, more preferably between 35 and 55 mm, whereby said sleevelike or pocketlike portion has a length of at most 60 cm, whereby said first and second position are spatially separated over a predetermined distance, preferably at least 20 cm, more preferably at least 25 cm, even more preferably at least 30 cm, most preferably at least 35 cm, said predetermined distance preferably at most 60 cm, more preferably at most 55 cm, even more preferably at most 50 cm.

28. Surgical drape according to claim 27, wherein said through-hole and sleevelike or pocketlike portion are adapted to receive a first elongate element, whereby the sheet optionally comprises a removable cover for covering the through-hole, most preferably whereby the maximal diameter of the sleevelike or pocketlike portion is about equal to the diameter of the through-hole.

29. Surgical drape according to claim 27, wherein said sleevelike or pocketlike portion comprises a material suitable for allowing a second element to embrace the distal end of the first elongate element from the distal surface.

30. Surgical drape according to claim 27, whereby the sleevelike or pocketlike portion comprises a transparent or translucent material.

31. Surgical drape according to claim 27, wherein the sheet comprises at least one anchoring zone at a third, internal position on the proximal surface of the sheet adapted for positioning said anchoring zone on the proximal surface to a biometric anchoring point or zone on the body of the surgical patient, preferably to or around the knee or patella or alternatively at or around the navel, whereby said third position is spatially separated from the first position over a predetermined distance, said predetermined distance between the first and third position being dependent on the biometric anchoring point or zone, and whereby said third position is spatially separated from the second position over a predetermined distance, said predetermined distance being dependent on an averaged distance between the area to be operated on and the biometric anchoring point, preferably whereby the sheet at least partially transparent, translucent or open at or around the anchoring zone.

32. Surgical drape according to claim 31, whereby the predetermined distance between the first and third position is comprised between 25 cm and 55 cm, preferably between 30 cm and 50 cm, more preferably between 35 cm and 48, and most preferably between 40 cm and 45 cm.

33. Surgical drape according to claim 31, whereby the second position is not substantially aligned with a line defined by the first and third position, and preferably distanced at least 20 cm, more preferably at least 25 cm, from said line.

34. Surgical drape according to claim 27, wherein the sheet, not considering transparent, translucent or open portions and the sleevelike or pocketlike portion, substantially comprises three layers, at least one of said layers, preferably at least the layer at the proximal surface, being waterproof, at least one of said layers, preferably at least the layer at the distal surface, being liquid-absorbent, and at least one of said layers, preferably at least the middle layer and optionally the layer at the distal surface, being reinforcing.

35. Surgical drape according to claim 27, whereby the surgical sheet is essentially rectangular, and comprises a central sheet portion wherein the incision window and through-hole are positioned, said central sheet portion, not considering transparent, translucent or open portions and the sleevelike or pocketlike portion, substantially comprising at least two layers, preferably three layers, and whereby the sheet furthermore comprises a fringe sheet portion around the central sheet portion which comprises less layers than the central sheet portion.

36. Surgical drape according to claim 27, comprising an anchoring zone, whereby the surgical drape is suitable for use in hip surgeries, preferably whereby the distance between the incision window and the anchoring zone is suitable for positioning the anchoring zone at or around the patella or knee, or at or around the navel of a patient.

37. Surgical drape according to claim 27, whereby the sheet is mirror symmetric with respect to a central line along the length of the sheet, each symmetric side comprising at least one of said transparent or open incision windows, and at least one, preferably one, of said through-holes and sleevelike or pocketlike portion, and preferably whereby each symmetric side comprises at least one of said anchoring zones.

38. Method for unfolding and positioning a folded surgical drape according to claim 27, on a body of a surgical patient which is located on an operating table, said table comprising at least one lateral, movable, substantially vertically extending, mounting element for receiving modular surgery instruments, said method comprising the following steps:
a. positioning the incision window over the area of the body to be operated on, preferably by affixing said incision window around said area via an adhesive portion on the proximal surface of the sheet at or around the incision window;
b. placing the through-hole and the sleevelike or pocketlike portion over the mounting element;
c. unfolding the sheet, thereby covering the body of the surgical patient;
d. optionally placing a modular surgery instrument on the mounting element;
whereby the step of positioning the incision window over the area to be operated on precedes the step of placing the through-hole and the sleevelike or pocketlike portion over the mounting element.

39. Method for unfolding and positioning a surgical drape according to claim 38, whereby the surgical drape furthermore is a surgical drape, wherein said method further comprises the following step:
positioning, preferably affixing, the anchoring zone to a biometric anchoring point or zone on the body of the patient;
whereby the step of positioning the anchoring zone to the biometric anchoring point or zone precedes the step of positioning the incision window over the area to be operated on.

40. Method for unfolding and positioning a surgical drape according to claim 37, on a body of a surgical patient which is located on an operating table, said table comprising at least one lateral, movable, substantially vertically extending, mounting element for receiving modular surgery instruments, said method comprising the following steps:
a. positioning the incision window on the first lateral symmetric side of the sheet over the area of the body to be operated on, preferably by affixing said incision window around said area via an adhesive portion on the proximal surface of the sheet at or around the incision window;
b. positioning the incision window on the second lateral symmetric side of the sheet over an area on the body mirrored to the area of the body to be operated on, preferably by affixing said incision window around said mirrored area via an adhesive portion on the proximal surface of the sheet at or around the incision window;
c. placing the through-hole and sleevelike or pocketlike portion on the first lateral symmetric side of the sheet over the mounting element most proximal to said first lateral symmetric side of the sheet;
d. placing the through-hole and sleevelike or pocketlike portion on the second lateral symmetric side of the sheet over the other mounting element;
e. unfolding the sheet, thereby covering the body of the surgical patient;
f. optionally placing modular surgery instruments on one or both of the mounting elements;
whereby the steps of positioning the incision window over the area to be operated on precede the steps of placing the through-hole and sleevelike or pocketlike portion over the mounting element.

41. Method for unfolding and positioning a surgical drape according to claim 40, whereby the surgical drape furthermore is a surgical drape, wherein said method further comprises the following step:
affixing a first of said anchoring zones of the sheet to the biometric anchoring point or zone on a first lateral side of the body of the patient;
affixing a second of said anchoring zones to the biometric anchoring point or zone on the second lateral side of the body of the patient;
whereby the steps of affixing the anchoring zones to the biometric anchoring points or zones precede the steps of positioning the incision window over the area to be operated on.

42. Method for unfolding and positioning a surgical drape according to claim 41, whereby the surgical drape is a surgical drape, and wherein the anchoring zone is provided to the sheet internal to a flap, said flap being foldably attached to said sheet, wherein:
the step of affixing a first of said anchoring zones of the sheet to the biometric anchoring point or zone on a first lateral side of the body of the patient, is preceded by the following step:
unfolding the flap comprising the first anchoring zone from the folded sheet to overlay the biometric anchoring point or zone on the first lateral side of the body of the patient;
the step of affixing a second of said anchoring zones to the biometric anchoring point or zone on the second lateral side of the body of the patient, is preceded by the following step:
unfolding the flap comprising the second anchoring zone from the folded sheet to overlay the biometric anchoring point or zone on the second lateral side of the body of the patient.

43. Method for folding up a surgical drape according to claim 27, said sheet being essentially rectangular and having a head side, a leg side, and two lateral sides, comprising the following steps:
a. folding up a section of the sheet on the lateral side of said sheet most proximal to the through-hole, over the sheet substantially up to, but not over, the incision window, and whereby said folded section preferably comprises said through-hole and the associated sleevelike or pocketlike portion, more preferably whereby the folded section on the lateral side is accordion folded;
b. folding up a section of the sheet on the leg side, preferably at least partly, more preferably at most partly or alternatively entirely, over the incision window, preferably whereby the folded section on the leg side is accordion folded, more preferably whereby the folded section on the leg side is doubly accordion folded;
c. folding up the section of the sheet on the head side which is not folded over with the folded leg side section, preferably whereby the folded section on the head side is accordion folded, more preferably whereby the folded section on the head side is at least doubly, even more preferably at least triply or quadruply accordion folded.

44. Method for folding up a surgical drape according to claim 43, whereby the surgical drape is a surgical drape, whereby the method comprises a final folding step of folding the sheet over the central symmetry axis onto itself.

45. Method for folding up a surgical drape according to claim 43, whereby one or more folding steps is followed by a step of providing one or more seals attaching the folded sections to another part of the sheet.

46. The use of a surgical drape according to claim 27 for the sterile shielding of a patient during a surgical procedure, preferably hip surgery, more preferably anterior approach hip surgery.
